(19) United States
(12) Patent Application Publication
(10) Pub. No.: US 2014/0127710 A1
(43) Pub. Date: May 8, 2014

(54) BACKGROUND-FREE MAGNETIC FLOW CYTOMETRY

(71) Applicants: Oliver Hayden, Herzogenaurach (DE); Michael Johannes Helou, Regensburg (DE); Mathias Reisbeck, Obertraubling (DE); Sandro Francesco Tedde, Erlangen (DE)

(72) Inventors: Oliver Hayden, Herzogenaurach (DE); Michael Johannes Helou, Regensburg (DE); Mathias Reisbeck, Obertraubling (DE); Sandro Francesco Tedde, Erlangen (DE)

(73) Assignee: Siemens Aktiengesellschaft, Munchen (DE)

(21) Appl. No.: 14/128,605

(22) PCT Filed: Jun. 12, 2012
(86) PCT No.: PCT/EP2012/061108

§ 371 Date: Dec. 21, 2013

(30) Foreign Application Priority Data
Jun. 21, 2011 (DE) 102011077905.1

(51) Int. Cl. G01N 15/14; G01N 33/543

(52) U.S. Cl. CPC G01N 15/1404; G01N 33/54326
USPC 435/7.2; 435/287.1; 29/890.09

(57) ABSTRACT

The invention relates to an apparatus and a method for magnetic flow cytometry, wherein magnetic units (22, 24) are arranged in a flow channel (10) which is configured, with respect to the channel diameter (100) and the surface condition of the channel inner wall, in such a manner that a flow of a complex suspension can be produced in the flow channel (10) with a laminar flow profile (40). The forces (F_{M}) that can be caused by the magnetic units (22, 24) and the forces (F_{S}) that can be caused by the flow, applied to magnetic markers (26) that are not bound to cells, have the effect of holding back said magnetic markers (26) that are not bound to cells in the front channel section (240) and preventing them from continuing to flow along the flow channel (10) via the cell measuring device (20).

### BACKGROUND—FREE MAGNETIC FLOW CYTOMETRY

The present invention relates to flow cytometry, in particular magnetic cell measurement.

In the field of cell measurement and cell detection, besides optical measurement methods such as scattered-light or fluorescence measurement, magnetic detection methods in which the cell types to be detected are marked by means of magnetic labels are also known.

In particular, for magnet-based measurement, methods in which magnetically marked cells are separated by means of magnetophoresis from a complex cell suspension, for example a blood sample, are known. The magnetic marking is carried out in particular by introducing cell-specific markers into the complex cell sample. By means of magnetophoresis, magnetically marked cells, or generally magnetic particles, can be guided or directed in flow, and thereby separated.

For cell measurement in diagnosis and science, it is necessary in particular to measure cell types which are present only in very low concentrations in a blood sample, for example disseminated tumor cells. For the quantification of cell concentrations, or for reliable detection of particular cells, single-cell detection is therefore desirable. It is known that this requires prior enrichment of the cells to be determined from a suspension with a complex background. In most cases, however, this alone does not lead to sufficient specificity of the measurement. Precisely since magnetic cell measurement involves very simple sample preparation by addition of the cell-specific markers, in magnetic flow cytometry the problem arises that unbound magnetic markers always cause a background signal. This background leads, for example, to false-positive detection signals.

It is an object of the present invention to minimize a background signal due to unbound magnetic markers in magnetic flow cytometry.

The object is achieved by an apparatus as claimed in patent claim 1. A method for magnetic flow cytometry is specified in patent claim 9. A production method for the apparatus according to the invention is specified in patent claim 15. Advantageous configurations of the invention are the subject-matter of the dependent claims.

The apparatus according to the invention for magnetic flow cytometry comprises a flow channel, a first magnetic unit for enrichment and a second magnetic unit for alignment of magnetically marked cells, and at least one cell measuring device. The magnetic units are arranged in a front channel section, with respect to the flow direction. The flow channel is configured in respect of channel diameter and surface composition of the channel inner wall in such a way that a flow of a complex suspension in the flow channel can be generated with a laminar flow profile. The flow channel is furthermore configured in such a way that the forces exertable by the magnetic units and the forces exertable by the flow act on magnetic markers not bound to cells in such a way that these unbound magnetic markers can be retained in the front channel section. This has the advantage that these unbound markers do not reach the cell measuring device, which lies further back in the flow channel in the flow direction. The markers not bound to cells are therefore retained in the front channel section and do not flow over the cell measuring device in the further course of the channel, and are thus eliminated as a perturbing component so that the background signal which is caused by the unbound magnetic markers is avoided. This has the advantage that a higher specificity is ensured in the measurement of the magnetically marked cells, in particular single-cell detection.

In particular, the first magnetic unit in the apparatus is arranged in the front channel section in such a way that a gradient magnetic field, which enriches magnetically marked cells and magnetic markers not bound to cells on the channel bottom inside the flow channel, can thereby be generated. The enrichment has the advantage of bringing the magnetically marked cells close to the channel bottom for measurement at the cell measuring device, and furthermore has the advantage that magnetic markers not bound to cells are brought onto the second magnetic unit at the channel bottom, by which, as will be described below, retention of the unbound markers is preferably favored.

In particular, this second magnetic unit is arranged in the front channel section in such a way that magnetically marked cells are thereby aligned inside the flow channel along an axis on which the cell measuring device is arranged in the further course of the flow channel. This arrangement of the second magnetic unit has the advantage that magnetophoretic guiding of the magnetically marked cells can be carried out, by which the cells can be aligned and, in particular, guided individually over the cell measuring device.

This second magnetic unit is, for example, furthermore arranged on the channel bottom in such a way that it protrudes into the flow channel. This has the advantage that, in addition to the magnetic force on the magnetic markers, particularly the unbound magnetic markers, the magnetic unit can also induce a mechanical hindrance to further flow of these magnetic markers. As an alternative, the guide strips could also be formed in the channel bottom so that they do not represent a mechanical obstacle for the flow.

In this case, however, the magnetic holding force which acts on the unbound markers must be greater or the flow rate must be less, in order to be able to reliably retain the unbound magnetic markers equally.

The second magnetic unit has, in particular, magnetic guide strips. These are made, in particular, of a ferromagnetic material. Preferably, these magnetic guide strips are arranged in a herringbone pattern. The guide strips thus point in the shape of an arrow at the middle of the channel bottom. The magnetically marked cells are therefore aligned particularly effectively on this central axis along the channel bottom, where they then flow onto the cell measuring device. In order to fulfill the filter function, in particular, the guide strips extend over the entire channel width.

In an advantageous embodiment of the invention, the second magnetic unit in the apparatus is configured in such a way that a magnetic force and an additional retaining force can be exerted by this second magnetic unit on the magnetic markers not bound to cells, these forces counteracting in direction and magnitude those of the shear force of the flow of the complex suspension. This configuration of the magnetic unit thus has the advantage of a combination of two forces on the unbound markers, by means of which this can be retained against the flow direction in the channel.

In another advantageous configuration of the invention, the flow channel of the apparatus is configured in respect of the channel diameter in such a way that cell aggregates of a plurality of cells, which are bound to one another by means of magnetic markers, can protrude into the middle of the channel to such an extent that, owing to the forces exertable on the cell aggregates, these cell aggregates can be transported away by the flow rate prevailing in the middle of the channel. This configuration in respect of the channel diameter thus offers the further advantage that cell aggregates also do not lead to false-positive signals since they are transported away by the highest flow rate prevailing in the channel. In particular, the flow channel is furthermore configured in respect of the channel diameter in such a way that a distance from the cell measuring device which is arranged in particular on or in the channel bottom, at which no detection of the cell aggregate can be induced, can be maintained by cell aggregates which flow in the middle of the channel. That is to say, the channel diameter is selected to be so large that the cell aggregates of a plurality of cells, which are bound to one another by means of magnetic markers, flow past the cell measuring device at a sufficiently large distance therefrom. The sensitivity of a magnetoresistive sensor, for instance, decreases with 1/d3, d standing for the distance from the sensor. The cell measuring device is expediently produced with a magnetoresistive sensor. This may, in particular, be a GMR (giant magnetoresistance) sensor. It is advantageous to arrange a plurality of sensor elements, which are for example bridge elements of a Wheatstone bridge circuit.

In the method according to the invention for magnetic flow cytometry, a laminar flow of a cell sample with magnetically marked cells and magnetic markers not bound to cells is generated. Furthermore, the magnetically marked cells and the magnetic markers not bound to cells are dynamically enriched in a gradient magnetic field. In addition, the magnetically marked cells are magnetophoretically aligned along an axis. The magnetic field strength of the gradient magnetic field and the flow rate are selected in such a way that the forces acting on the magnetic markers not bound to cells retain these markers in the flow. This has the advantage that each retained unbound marker cannot contribute to a background signal.

In the method, in particular, the magnetic markers are added to the cell sample in excess. Although this entails a high background signal, it ensures for the first time that very specific cells, which for instance are present only in a low concentration in a sample, can be marked reliably without further sample preparation and can accordingly be detected selectively. Only by the concept of filtering out, or retarding, the excess magnetic markers can satisfactory specific single-cell detection be achieved in cell samples, for instance blood.

In an advantageous configuration of the invention, in the method the generation of the laminar flow of the cell sample is carried out in a flow channel, the dynamic enrichment takes place in the direction of the channel inner wall of the channel bottom, and the magnetophoretic alignment takes place along an axis, the axis extending in the flow direction along the channel inner wall of the channel bottom. By virtue of this axial profile, the magnetically marked cells are guided over a cell measuring device. The cell sample is in this case guided past a magnetic unit on the channel inner wall of the channel bottom in such a way that the magnetic markers in this cell sample which are not bound to cells are retained at precisely this magnetic unit.

Preferably, superparamagnetic markers are used as magnetic markers in the method.

In an advantageous embodiment of the method, the magnetic field strength of the gradient magnetic field and the flow rate are selected in such a way that the effect of the forces acting on cell aggregates of a plurality of cells, bound to one another by means of magnetic markers, is that these cell aggregates are transported away by the flow rate prevailing in the middle of the channel. This has the further advantage that the cell aggregates cannot lead to false-positive signals either. In particular, the middle of the channel, where the cell aggregates move, is so far from the cell measuring device, in particular the magnetoresistive sensor on or in the channel wall, that the stray magnetic field of the markers in or around the cell aggregates is not detected.

Thus, in particular, in the method a cell sample is injected into an embodiment of the apparatus described above.

In the production method according to the invention for an apparatus for magnetic flow cytometry, the second magnetic unit for alignment of magnetically marked cells is arranged on the channel bottom in the flow channel and, in particular, protrudes into the flow channel. This has the advantage over arrangement in the channel bottom of also producing a mechanical retaining force due to a flow obstacle, in addition to the magnetic holding force.

One particular advantage of the present invention is that the simplicity of the sample preparation for the magnetic flow cytometry is preserved by virtue of the retardation of unbound markers in order to reduce the background signal. This is, in particular, an essential advantage of magnetic measurement. Since an excess of magnetic markers must be added to the sample for sufficiently reliable marking of the cells to be detected, this reduction of the background is of even more essential importance for an improvement of the signal-to-noise ratio. In particular, superparamagnetic labels that comprise antibodies, by means of which the superparamagnetic labels can bind selectively to the isotopes on the cell surface, may be envisioned as magnetic markers. Then, for example, one superparamagnetic nanoparticle is respectively bound to the antibodies. The nanoparticles have, in particular, diameters of between 20 and 200 nm.

Magnetic labels are typically very small. If they are not bound to cells, they have hydrodynamic diameters of less than 500 nm. Once enriched on the channel bottom, small magnetic units of this type can therefore be retarded well by means of the magnetic forces, particularly since the lowest flow rates prevail at the channel bottom. Selected flow rates are typically less than 5 mm/s. In contrast to the unbound markers, marked cells or larger magnetic beads have diameters of for example from 3 to 20 μm, and cell aggregates correspondingly have even larger hydrodynamic diameters. The further the particles reach into the microfluidic channel, the higher the flow rate is and therefore the greater is the extent to which they are entrained by the laminar flow.

The magnetic unit for magnetophoretic alignment of the magnetically marked cells advantageously has a herringbone structure. Such structures have proven particularly effective for aligning magnetically marked cells two-dimensionally on a channel bottom, in such a way that they travel individually in succession along an axis and can therefore be guided individually over a sensor unit, for example a magnetoresistive sensor. The cell measuring device is, for example, configured as a Wheatstone bridge circuit and has at least one magnetoresistive sensor, in particular a plurality of magnetoresistive sensors, as bridge element(s).

Furthermore, the herringbone structure is not unsuitable for also constituting a correspondingly mechanical obstacle for the flow of the unbound magnetic particles, and thereby further reinforcing the retardation. In particular, the “herringbones” of the magnetic unit cover the entire channel width.

The flow channel is, in particular, a microfluidic channel. The diameter of the channel is, in particular, adapted to the respective cell sample. The characteristic cell diameter, which is moreover important for the influence of the flow profile on cells and particles of the suspension, varies according to the type of cell to be detected.

One essential component of the method is thus, in particular, controlled dynamic enrichment of cells in a small suspension volume. The enrichment is carried out in the direction of the microfluidic bottom by means of an external magnet. Besides the type of magnetic markers and their stray magnetic field, the flow rate and the microfluidic dimensioning, the essential parameters for stringent enrichment of the magnetically marked cells are furthermore the configuration of the magnetophoretic guide lines, for example their angle with respect to the flow direction and their magnetic moment, as well as the external gradient magnetic field.

By virtue of the advantageous use of magnetophoresis of the herringbone type, single-cell detection is ensured by the enrichment taking place in three dimensions and also the simultaneous in-situ filtering of unbound markers from the suspension. Constraints for the in-situ filtering are the retaining forces of the ferromagnetic lines, the external magnetic field, and also the flow rate and the hydrodynamic diameter of the markers in relation to the hydrodynamic diameter of the analyte, i.e. the magnetically marked cell or, for instance, a magnetic bead.

Combination of the magnetic retardation of the unbound markers with the filtering of the markers at the ferromagnetic lines, which extend in particular over the entire width of the channel bottom, is particularly advantageous. In this way, the unbound markers cannot flow past these mechanical obstacles at any point without their having to counteract the external magnetic field. In this way, dynamic filtering of the unbound markers is ensured.

In an alternative embodiment, the ferromagnetic guide strips are arranged, for example, in such a way that they begin at the channel walls on both sides and converge obliquely toward the middle of the channel, for example at an angle of between 0° and 90° relative to the channel wall. In this case, the guide strips point in particular in the direction of the flow direction. In the middle of the channel, however, the guide strips do not touch as in the case of the herringbone structure, but engage in one another with a slight offset.

Furthermore, the magnetophoresis may for example also be preceded by further ferromagnetic strips as filter strips. That is to say, before the magnetophoresis in the flow direction, ferromagnetic filter strips extend transversely over the channel bottom from one channel wall to the other. They may be arranged perpendicularly or at any desired angle of between 0° and 90° with respect to the channel walls.

The described magnetic flow cytometry apparatus has the additional particular advantage that its filter effect can be replenished after use by regenerating the cytometer. To this end, in particular, the external magnetic field which is induced by the first magnetic unit can be removed or turned off. In addition, flushing with a very high flow rate, which washes out the filtered particles, may be carried out.

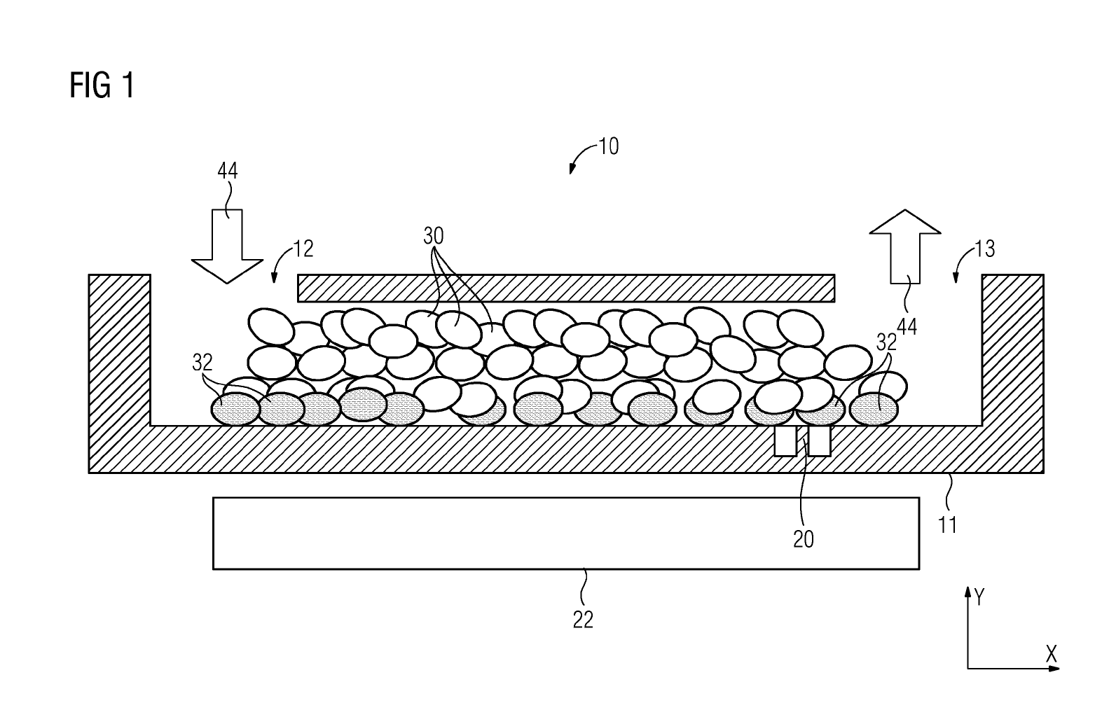

Embodiments of the present invention will be described by way of example with reference to FIGS. 1 to 6 of the appended drawing:

FIG. 1 shows a cross section through the flow channel of the apparatus,

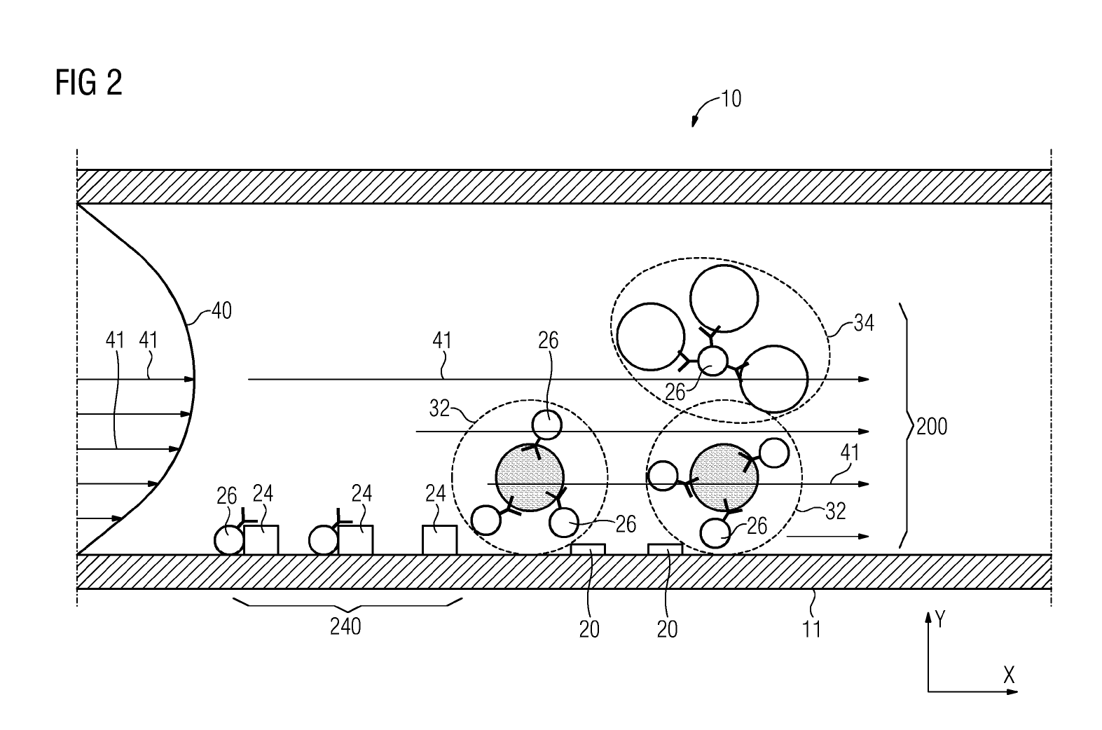

FIG. 2 shows a detail of the cross section through the flow channel with the arrangement of the magnetic guide lines and the flow profile,

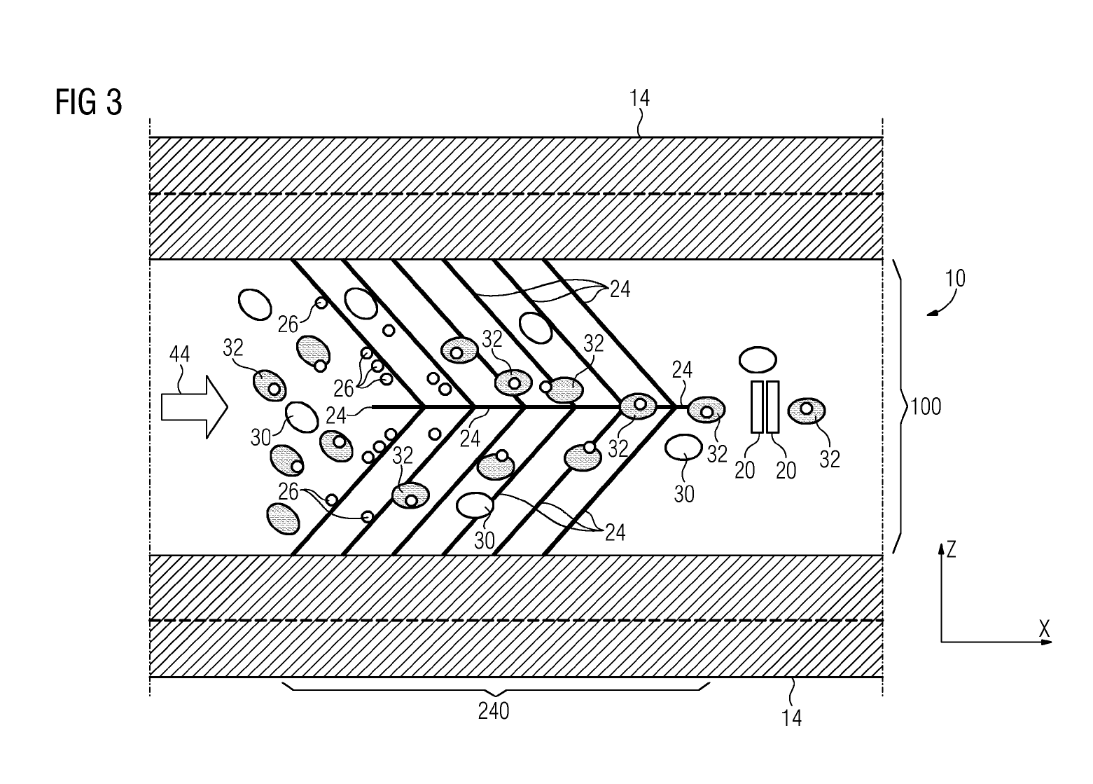

FIG. 3 shows a plan view of the arrangement of the magnetic guide lines,

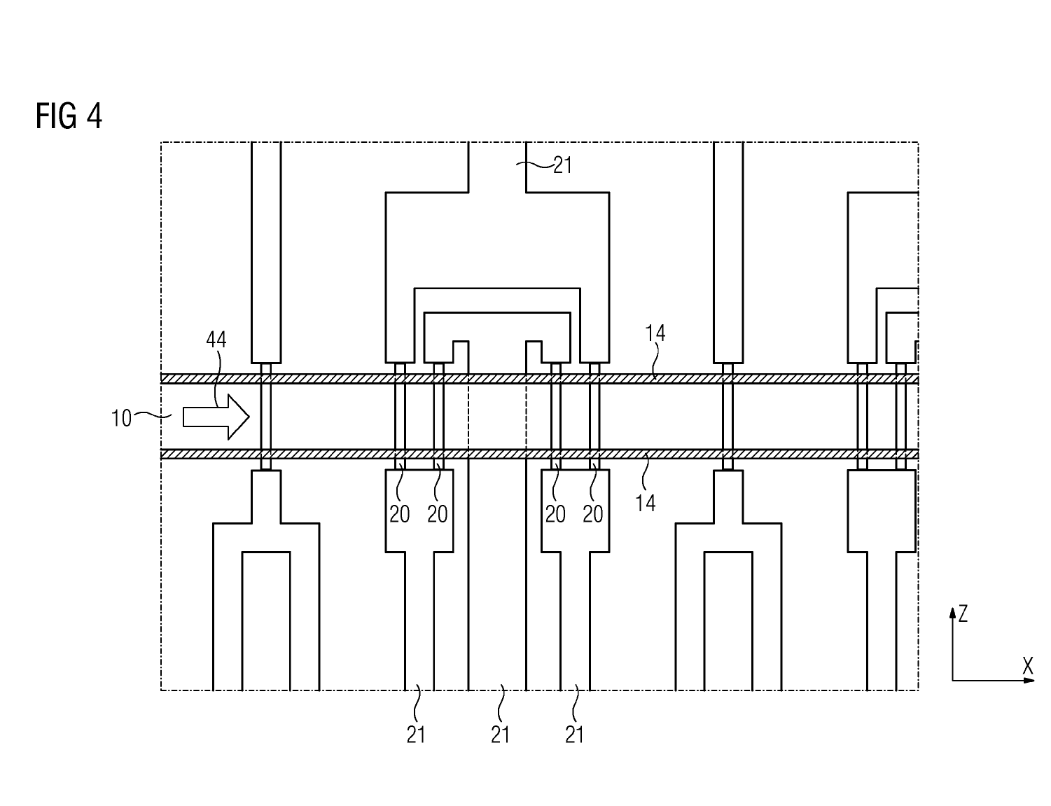

FIG. 4 shows a plan view of the arrangement of the sensor units with the flow channel,

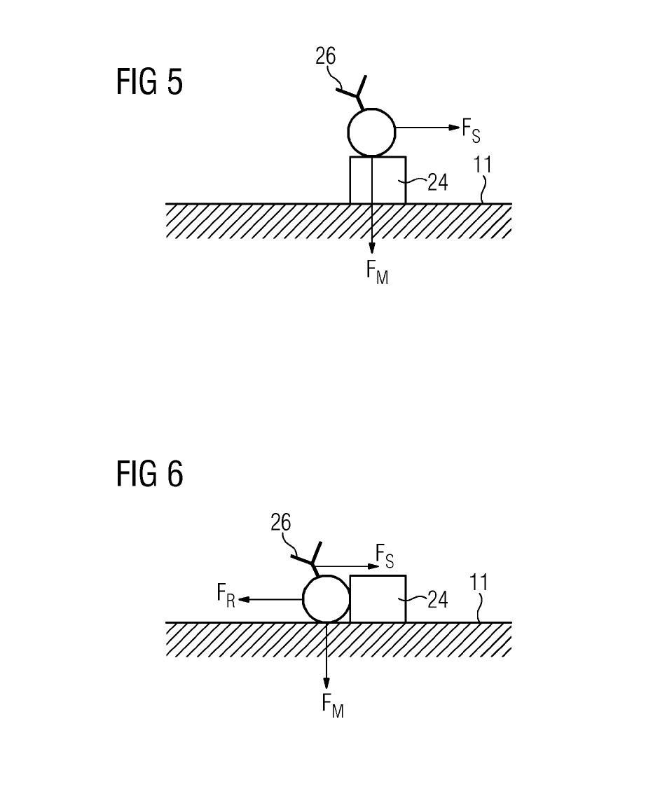

FIG. 5 shows a first example of a force distribution on an unbound magnetic marker and

FIG. 6 shows another example of a force distribution on an unbound magnetic marker.

FIG. 1 shows a cross section through a schematic representation of a flow channel 10. It has an upper boundary and a channel bottom 11. A channel inlet 12 is shown on the left-hand side and a channel outlet 13 is shown on the right-hand side. The arrows 44 indicate the flow direction. Two rectangles are indicated in the channel bottom 11 and represent the cell measuring device, i.e. the magnetic sensors 20. Below the channel bottom 11, a permanent magnet 22 is indicated along the entire channel length. It may, however, only amount to half the length and be restricted only to the front left channel section. The cells 30, 32 are indicated in the form of ellipses in the channel 10. In the drawing, unmarked cells 30 are distinguished from marked cells 32 by different shading. Only the magnetically marked cells 32 experience a magnetic force in the gradient field due to the permanent magnet 22 and are enriched on the channel bottom 11, so that they move over the magnetic sensor 20 in proximity thereto. All other cells 30 move over the sensor 20 substantially further away from the channel bottom 11. The length of the enrichment path 240, and therefore also the length of the permanent magnet 22 below the flow channel 20, must be selected according to the flow rate 41, the channel diameter and magnetic moment of the cell marking 26, in order as far as possible to enrich all magnetically marked cells 32 from the suspension on the channel bottom 11. The magnetic flow cytometry apparatus shown therefore involves a dynamic measurement, which is also preceded by dynamic enrichment of the cells 32. The dynamic measurement, in combination with the dynamic enrichment and the simple sample preparation, which essentially consists only in the magnetic markers 26 having to be added to the cell sample, is one of the great advantages of magnetic flow cytometry in comparison with other measurement methods in cell diagnosis, for instance fluorescence flow cytometry.

FIG. 2 shows a detail of the cross section through the flow channel 10. The flow profile 40 is schematically indicated on the left-hand side. In the case of a laminar channel flow 40, an essentially parabolic profile is set up. The arrows 41 represent the flow rates, which decrease from the middle toward the edge of the channel 10. The highest flow rate 41 thus prevails at the center of the channel.

The so-called enrichment and alignment path 240 is shown in the left-hand region of the channel section. It thus precedes the detection region 20 in the flow direction 44. The magnetic units, the permanent magnet 22 and the magnetic guide lines 24 are thus arranged in this front channel section 240. The magnetic guide lines 24 are in particular ferromagnetic metal strips, for example of nickel. In FIG. 2, these strips 24 are placed on the channel bottom 11 in such a way that they protrude into the flow channel 10. The magnetic sensors 20, over which the magnetically marked cells 32 are guided, are shown after this front channel section 240 in the flow direction 44.

The magnetically marked cells 32 are again represented by shading. It is, however, necessary to ascertain whether the cell is so to speak a correctly magnetically marked cell 32 which has a plurality of magnetic markers 26 and moves as an individual marked cell 32 in the complex suspension, or whether cells 30 are incorrectly attached to an individual magnetic marker 26 and are aggregated by means of this. Such an aggregate of a plurality of cells 34, which are bound to one another by means of magnetic markers 26, has a substantially greater hydrodynamic diameter than an individual marked cell 32. This is crucial for the different flow behaviors of individual cells 32 and cell aggregates 34. Owing to the substantially greater hydrodynamic diameter, such an aggregate 34 always extends very much further into the middle of the channel where the higher flow rate 41 prevails. The large aggregates 34 are entrained by this high flow rate 41 and are therefore in turn moved further away from the channel bottom 11, so that they flow past the sensor 20 at too great a distance 200 therefrom and therefore cannot be detected. This therefore avoids false-positive signals due to cell aggregates 34. In this way, the cell measurement can thus be made specific by parameters such as the flow channel diameter and the flow profile 40, or the flow rate 41. The sensitivity of the sensor 20 decreases with 1/d3, where d stands for the distance from the sensor.

The perturbing background signal during the measurement is essentially caused by unbound markers 26, which are added in excess to the cell sample in order to ensure complete marking of all cells 32 to be detected in the sample. The magnetic markers 26 are, for example, superparamagnetic labels which bind to isotopes on the cell surface by means of antibodies. The magnetosensors 20 are for example, GMR sensors, GMR standing for Giant MagnetoResistance.

FIG. 2 schematically shows the filter principle of the device. The small unbound markers 26 have only very small hydrodynamic diameters, and come in proximity to the channel bottom 11 owing to the magnetic enrichment. There, they can be so to speak filtered out of the flow and stopped by the ferromagnetic strips 24. In this case, the ferromagnetic strips 24 firstly act as a mechanical obstacle in the flow. The magnetic markers 26 would need to move against the gradient magnetic field of the permanent magnet 22 in order to escape from the magnetic filter. In addition, magnetic holding forces FM are furthermore created at these ferromagnetic strips 24, and retard the magnetic markers 26. The filtration is thus a combination of a magnetic force FM filter and a shear force FS filter.

FIG. 3 now shows a plan view of the channel detail shown in FIG. 2. The flow direction 44 is again denoted by an arrow. The enrichment path 240 is again shown in the front, i.e. front in the flow direction, region of the channel 10. The ferromagnetic guide lines 24 for the magnetophoretic enrichment and alignment of the magnetically marked cells 32 extend through this region. The magnetic guide lines 24 are arranged in a particularly advantageous herringbone pattern which converges acutely from the channel walls 14 to the middle of the channel. In this case, it is particularly advantageous for an effective filter for the unbound magnetic markers 26 that the magnetic guide strips 24 cover the full channel width 100 and do not leave any gaps. The permanent magnet 22, which is not explicitly shown in the figure since it lies below the channel bottom 11, also extends in particular over the entire channel width 100 so that a uniform gradient field acts over the entire channel width 100 on the magnetic particles 26 in the suspension. It is particularly advantageous for the permanent magnet 22 to extend beyond the channel width 100, for example as far as the dashed line which runs through the channel wall 14, so that it induces a uniform gradient field inside the channel 10.

A centrally extending magnetic guide line 24, which marks the middle of the channel and may be regarded as the described axis on which the magnetically marked cells 32 are aligned, is furthermore shown in FIG. 3. The magnetic sensors 20, over which the magnetically marked cells 32 flow, then lie in imaginary extension of this axis in the flow channel 10. FIG. 3 also shows unmarked cells 30, which are not influenced by the magnetic measures.

Instead of magnetically marked cells 32, magnetic beads may also be enriched and aligned in this way. Other analytes which can be magnetically marked may also be envisioned for such a measurement method.

In fluorescence flow cytometry as well, fluorescent markers are added in excess in the sample preparation phase, and then need to be separated by centrifuging and washing steps. Such steps are not necessary for magnetic flow cytometry when the parameters of the magnetic units and of the flow behavior are adjusted suitably to the size of the cell to be detected.

Lastly, FIG. 4 shows the arrangement of the magnetic sensor unit with the magnetoresistive elements 20, which are connected to one another in a Wheatstone bridge circuit. In this case, electrical supply lines 21 to the magnetoresistors 20 are also shown. The arrow again indicates the flow direction 44 through the flow channel 10.

Lastly, FIGS. 5 and 6 are intended to represent the forces acting on the unbound magnetic markers 26, again schematically. To this end, only a detail of the channel bottom 11 with a ferromagnetic strip 24 is shown. In FIG. 5, a magnetic marker 26 is shown with an antibody and a magnetic particle, which is held by means of the magnetic holding force FM on the channel bottom 11 as a result of the ferromagnetic strips 24 together with the gradient field of the permanent magnet 22, which lies below the channel bottom 11. This magnetic force FM acts on the magnetic marker 26 perpendicularly in the direction of the channel bottom 11, and retains it on the strip 24. However, the shear forces FS of the flow of the complex suspension also act on the magnetic marker 26. These forces act parallel to the channel bottom 11, i.e. in the flow direction 44. The magnetic holding forces FM must therefore be greater than the shear force FS in order to retain the magnetic marker 26.

FIG. 6 lastly shows that the arrangement of the ferromagnetic strip 24 on the channel bottom 11 also contributes to the filtering. The ferromagnetic strip 24 protrudes into the flow channel 10 in such a way that magnetic markers 26 can be trapped behind the ferromagnetic strips 24 in the flow direction 44. There, the magnetic force FM of the permanent magnet 22, which lies below the channel bottom 11, acts perpendicularly to the channel bottom 11 on the magnetic marker 26. The applied shear force FS of the flow 44 of the complex suspension thus already has a smaller interaction surface of the magnetic marker 26 available. In addition, the ferromagnetic strip 24 presents a flow obstacle, which means an additional retaining force FR for the magnetic marker 26.

In a flow channel 10 having a cross-sectional area of 0.105 μm2 with a throughput of about 1 μl/s, the flow rate 41 is about 1500 μm/s and the complex suspension passes over the magnetic sensors 20. The nickel strips 24 retain the magnetic nanobeads 26. After the measurement, these nanobeads 26 need to be removed from the nickel strip system 24. To this end, the flow channel 10 is flushed with a flow rate of for example 4 μl/s. The unbound magnetic nanobeads 26 then also move out of the filter 24 through the channel 10. In addition to the higher flow rate, to this end the external magnetic field may be minimized or turned off.

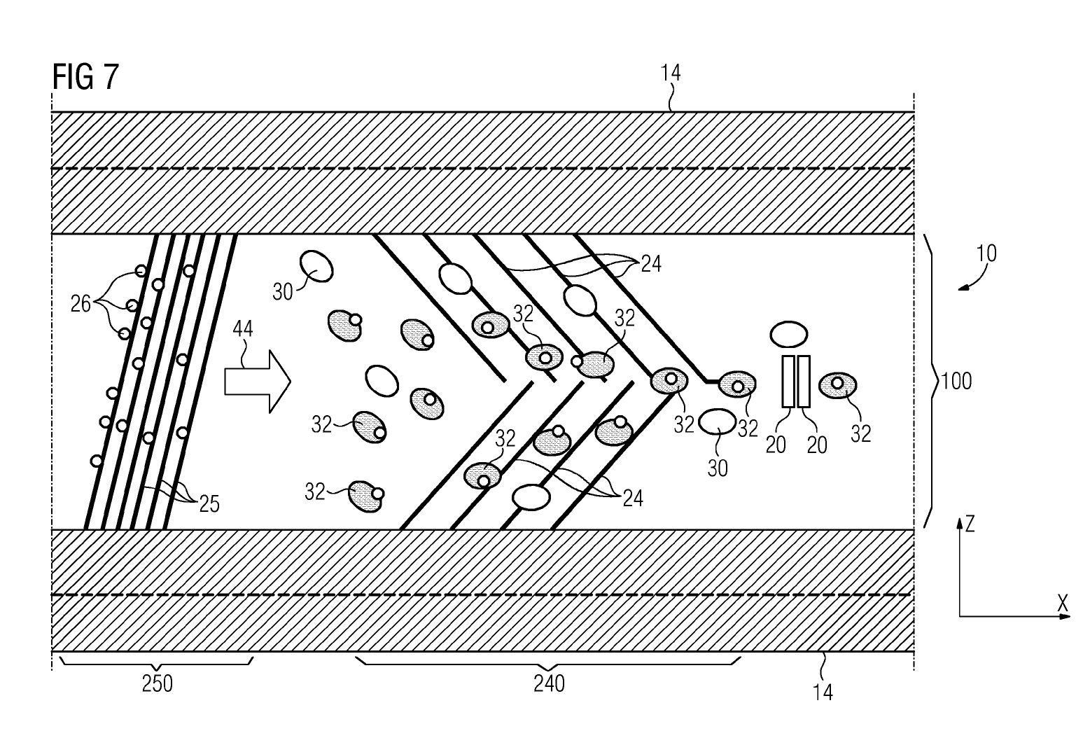

Lastly, FIG. 7 shows another alternative embodiment of the channel 10, with offset ferromagnetic guide strips 24 which do not touch in the middle of the channel 10 but engage in one another in the manner of a zipper. They are also preferably arranged at an angle of around 45° with respect to the channel walls 14 and point in the direction of the flow direction 44. Regardless of the precise configuration, the magnetophoresis 240 may also be preceded by an additional filter 250. This is thus arranged further forward in the channel 10 in the flow direction 44, further to the left in FIG. 7. To this end, ferromagnetic strips 25 extend transversely over the channel bottom 11 from one channel wall 14 to the other. They are, in particular, arranged perpendicularly or at an angle of between 0° and 90° with respect to the channel walls 14.

1. An apparatus for magnetic flow cytometry, having
a flow channel (10),
a first magnetic unit (22) for enrichment and
a second magnetic unit (24) for alignment of magnetically marked cells (32), and
at least one cell measuring device (20),
wherein the magnetic units (22, 24) are arranged in a front channel section (240), with respect to the flow direction (44), and the flow channel (10) is configured in respect of channel diameter (100) and surface composition of the channel inner wall in such a way that a flow of a complex suspension in the flow channel (10) can be generated with a laminar flow profile (40), so that the forces (Fs) exertable by the magnetic units (22, 24) and the forces (FS) exertable by the flow act on magnetic markers (26) not bound to cells in such a way that these magnetic markers (26) not bound to cells can be retained in the front channel section (240).

2. The apparatus as claimed in claim 1, wherein the first magnetic unit (22) is arranged in the front channel section (240) in such a way that a gradient magnetic field, which enriches magnetically marked cells (32) and magnetic markers (26) not bound to cells on the channel bottom (11) inside the flow channel (26), can thereby be generated.

3. The apparatus as claimed in claim 1 or 2, wherein the second magnetic unit (24) is arranged in the front channel section (240) in such a way that magnetically marked cells (32) are thereby aligned inside the flow channel (10) along an axis on which the cell measuring device (20) is arranged in the further course of the flow channel (10).

4. The apparatus as claimed in one of the preceding claims, wherein the second magnetic unit (24) for alignment of magnetically marked cells (32) is arranged on the channel bottom, particularly in such a way that it protrudes into the flow channel (10).

5. The apparatus as claimed in one of the preceding claims, wherein the second magnetic unit (24) has magnetic guide strips in particular ferromagnetic guide strips, the magnetic guide strips extending over the entire width of the channel bottom.

6. The apparatus as claimed in one of the preceding claims, wherein the second magnetic unit (24) for alignment of magnetically marked cells (32) is configured in such a way that a magnetic force (FM) and an additional retaining force (FR) can be exerted by this second magnetic unit (24) on the magnetic markers (26) not bound to cells, these forces counteracting in direction and magnitude those of the shear force (FS) of the flow of the complex suspension.

7. The apparatus as claimed in one of the preceding claims, wherein the flow channel (10) is configured in respect of the channel diameter (100) in such a way that cell aggregates (34) of a plurality of cells (30) bound to one another by means of magnetic markers (26) can protrude into the middle of the channel to such an extent that, owing to the forces (FM, FS) exertable on the cell aggregates (34), these cell aggregates (34) can be transported away by the flow rate (41) prevailing in the middle of the channel.

8. The apparatus as claimed in one of the preceding claims, wherein the flow channel (10) is configured in respect of the channel diameter (100) in such a way that a distance (200) from the cell measuring device (20) at which no detection of the cell aggregate (34) can be induced can be maintained by cell aggregates (34) of a plurality of cells (30), bound to one another by means of magnetic markers (26), which flow in the middle of the channel.

9. A method for magnetic flow cytometry, wherein
a laminar flow of a cell sample (40) with magnetically marked cells (32) and magnetic markers (26) not bound to cells is generated,
the magnetically marked cells (32) and the magnetic markers (26) not bound to cells are dynamically enriched in a gradient magnetic field,
the magnetically marked cells (32) are magnetophoretically aligned along an axis,
the magnetic field strength of the gradient magnetic field and the flow rate (41) being selected in such a way that the forces (FM, FS) acting on the magnetic markers (26) not bound to cells retain them in the flow.

10. The method as claimed in claim 9, wherein the magnetic markers (26) are added to the cell sample in excess.

11. The method as claimed in claim 9 or 10, wherein
the generation of the laminar flow of the cell sample (40) is carried out in a flow channel (10),
the dynamic enrichment takes place in the direction of the channel inner wall of the channel bottom (11), and
the magnetophoretic alignment takes place along an axis, the axis extending in the flow direction (44) along the channel inner wall of the channel bottom (11) so that the magnetically marked cells (32) are guided along this axis over a cell measuring device (20),
the cell sample (40) being passed over a magnetic unit (24) on the channel inner wall of the channel bottom (11) in such a way that the magnetic markers (26) not bound to cells are retained at this magnetic unit (24).

12. The method as claimed in one of claims 9 to 11, wherein superparamagnetic markers are used as magnetic markers (26).

13. The method as claimed in one of claims 9 to 12, wherein the magnetic field strength of the gradient magnetic field and the flow rate (41) are selected in such a way that the effect of the forces (FM, FS) acting on cell aggregates (34) of a plurality of cells (30) bound to one another by means of magnetic markers (26) is that these cell aggregates (34) are transported away by the flow rate (41) prevailing in the middle of the channel.

14. The method as claimed in one of claims 9 to 13, wherein a cell sample is injected into an apparatus as claimed in one of claims 1 to 8.

15. A production method for an apparatus as claimed in one of claims 1 to 8, wherein the second magnetic unit (24) for alignment of magnetically marked cells (32) is arranged on the channel bottom (11) in the flow channel (10), particularly in such a way that it protrudes into the flow channel (10).
